(19) United States
(12) Patent Application Publication
(10) Pub. No.: US 2018/0310502 A1
(43) Pub. Date: Nov. 1, 2018

(54) DELOSPERMA PLANT WITH A LARGE NUMBER OF PETALS AND A METHOD OF PRODUCING THE DELOSPERMA PLANT

(71) Applicant: Koichiro NISHIKAWA, Kanagawa (JP)

(72) Inventor: Koichiro NISHIKAWA, Kanagawa (JP)

(21) Appl. No.: 15/738,350

(22) PCT Filed: Dec. 21, 2016
(86) PCT No.: PCT/JP2016/089227

§ 371 Date: Dec. 20, 2017

(51) Int. Cl. A01H 5/02; A01H 3/00; A01H 6/30; A01H 1/04

(52) U.S. Cl. CPC A01H 5/02; A01H 3/00; A01H 5/06; A01H 1/04; A01H 6/30

(57) ABSTRACT

The Delosperma plant (1) comprises a flower with more than 150 petals, wherein the number of the petals consists of the number of outer petals (1a) and the number of inner petaloids (1b) around a pistil of the flower. This configuration shows that it is possible to produce the Delosperma plant (1) with a characteristic of having a large number of petals (more than twice as much) than conventional cultivars of Delosperma.

### BACKGROUND OF THE INVENTION

### 1. Field of the Invention

The present invention relates to a Delosperma plant which has a large number of petals and a method for producing the Delosperma plant.

### 2. Description of the Related Art

Delosperma belongs to the family Aizoaceae, and is native to South Africa in general. The name of the genus is derived from the Greek words delos (visible) and sperma (seed). Delosperma is an herbaceous perennial, and has succulent thick leaves. Its stem extends horizontally. Delosperma is widely used for open cultivation, container and landscape purposes in Japan owing to its cold and drought tolerance.

Delosperma usually has small flowers, which are similar to daisy flowers, while some Delosperma species have brightly-colored flowers and some other species bloom in spring and last until the beginning of winter. For example, Delosperma congestum, which is a kind of Delosperma species, has vivid yellow flowers and thick deep green leaves. This Delosperma congestum grows in colonies and gradually spreads out on the ground. And Delosperma cooperi (also known as its generic names HANARANZAN and REIKOU), which is an herbaceous perennial, has pink or crimson flowers and dense branches, and spreads quickly and its roots survive during winter.

In addition, there is a genus of plants similar to Delosperma, known as Lampranthus; however, this genus can be easily distinguished from Delosperma, because Lampranthus plants bloom only for a short period in spring, and the plants are sensitive and intolerant to cold.

The inventor of the present application has already bred many new cultivars of Delosperma plants and owns a plurality of plant patents in the U.S.. For example, the varietal denomination of the new cultivar bred by the inventor of the present application is ‘Jewel of Desert Rosequartz’ (refer to U.S. Plant Pat. No. 23,452); ‘Jewel of Desert Ruby’ (refer to U.S. Plant Pat. No. 23,453); ‘Jewel of Desert Garnet’ (refer to U.S. Plant Pat. No. 23,471); ‘Jewel of Desert Moon Stone’ (refer to U.S. Plant Pat. No. 23,491); ‘Jewel of Desert Topaz’ (refer to U.S. Plant Pat. No. 23,492); ‘Jewel of Desert Peridot’ (refer to U.S. Plant Pat. No. 23,566); ‘WOW(Wheels of Wonder)DRW5’ (refer to U.S. Plant Pat. No. 25,572); ‘WOWDRW7’ (refer to U.S. Plant Pat. No. 25,599); ‘WOWDOY3’ (refer to U.S. Plant Pat. No. 25,600); ‘WOWDAY2’ (refer to U.S. Plant Pat. No. 25,645) ‘WOW2011-1’ (refer to U.S. Plant Pat. No. 25,684); ‘WOWDRY1’ (refer to U.S. Plant Pat. No. 26,157); ‘DSAM13-1’ (refer to U.S. Plant Pat. No. 27,013); ‘DSAA13-1’ (refer to U.S. Plant Pat. No. 27,014); and ‘DSAB13-1.’ (refer to U.S. Plant Pat. No. 27,056). Note that the translated term of “Jewel of Desert,” which constitutes one of the names of new cultivars of Delosperma, has been registered as “SABAKU NO HŌSEKI (registered trademark)” in Japan.

Furthermore, the inventor of the present application has been providing these new cultivars of Delosperma to both several foreign and domestic corporations (refer to product catalogs available at Non-patent Reference 1 “Dümmen Orange Perennials 2017.” Retrieved Dec. 8, 2016, from https://issuu.com/dummenorange/docs/2017_catalog_Ir_perennials, Non-patent Reference 2; Delosperma “Jewel of Desert.” Retrieved Dec. 8, 2016, from http://www.delosperma.com/jewelofdesert/japanese.html, and Non-patent Reference 3; “Proven winners, Wheels of Wonder.” Retrieved Dec. 8, 2016, from https://www.soonerplantfarm.com/plant/ice-plant-wheels-of-wonder-fire-wonder/)

### BRIEF SUMMARY OF THE INVENTION

Conventionally, flowers that are more voluminous than a single flower, such as double flowers, with numerous petals look graceful and tend to enhance its commercial value since they are more attractive to consumers, even among plants of the same cultivar. For example, the inventions that increase the number of petals such of Verbena (EP 0995354 A2) and Calibrachoa (U.S. Pat. No. 7,786,342 B2) have succeeded in enhancing the value of each flower. Further, in response to the diverse needs of consumers today, it is more desirable for consumers to be able to select from among the various Delosperma varieties.

However, the first problem with conventional Delosperma is that it has relatively small numbers of petals so that the flowers look to be less voluminous. For example, Delosperma cooperi (Trailing Ice Plant) shown in FIG. 17, which has been widely cultivated, has approximately several dozen petals. Likewise, Delosperma plant shown in FIG. 18, which belongs to the varietal denomination of ‘Jewel of Desert Garnet’ (refer to U.S. Plant Pat. No. 23,471) has 64 petals (34 outer petals and 30 inner petaloids), and ‘Jewel of Desert Rosequartz’ (refer to U.S. Plant Pat. No. 23,452) has, for instance, 74 petals (44 outer petals and 30 inner petaloids).

There are some Delosperma species that have relatively large number of petals such as Delosperma sutherlandii and Delosperma ashtonii; however, these flowers have petals around 100 or less as shown in FIG. 19. In other words, conventional Delosperma species are characterized by relatively small numbers of petals, and the double-flower type species that have more petals—and therefore more commercially valuable—does not exist.

Next, the second problem with conventional Delosperma is that the number of blooms will be reduced if it generates seed through pollination. As a result, overall aesthetic of the flower has been diminished and hence this leads to the reduction of its commercial value. Accordingly, it is obvious that the development of new cultivar of Delosperma, which has flowers with many petals, together with no seed and has multiple blooms per stem, enhances its commercial value.

The present invention has been made in view of the abovementioned problems, and has an object to provide a Delosperma plant with a larger number of petals than conventional cultivars of Delosperma, and a method for producing the Delosperma plant. Furthermore, in addition to having a feature such that its flower has a large number of petals, the present invention also aims to provide a new cultivar of Delosperma with a large number of blooms.

### DISCLOSURE OF INVENTION

In order to solve the aforementioned issues, the present invention is a Delosperma plant comprising a flower with more than 150 petals, wherein the number of the petals consists of the number of outer petals and the number of inner petaloids around a pistil of the flower.

In this Delosperma plant, preferably, wherein the Delosperma plant includes more than 200 petals.

In this Delosperma plant, preferably, wherein the Delosperma plant includes more than 250 petals.

In this Delosperma plant, preferably, wherein the Delosperma plant includes more than 300 petals.

In this Delosperma plant, preferably, wherein the Delosperma plant comprises no stamen.

In this Delosperma plant, preferably, wherein the Delosperma plant comprises a female sterile flower, which is unable to generate seeds even when the pistil receives self or non-self pollens.

In this Delosperma plant, preferably, wherein the Delosperma plant forms no tuberous root.

In this Delosperma plant, preferably, wherein the Delosperma plant is (a) a cultivar called Variety A, which is produced by obtaining an individual mutant from hybridizing at least one of the individuals selected from a group consisting of Delosperma cooperi, Delosperma nubigenum, Delosperma coungestum, Delosperma esterhuysenisa, Delosperma floribundum, Delosperma aberdeenense and Delosperma ‘John Proffitt’, and by crossing using the individual mutant and then selecting said cultivar, (b) a cultivar produced by crossing species, wherein at least either one of the parents of the species is selected from the Variety A, or (c) a progeny of one of the cultivars (a) and (b).

In this Delosperma plant, preferably, wherein the Delosperma plant is (a) a cultivar called Variety B, which is produced by obtaining an individual mutant from hybridizing at least one of the individuals selected from a group consisting of ‘Jewel of Desert Rosequartz’ (U.S. Plant Pat. No. 23,452), ‘Jewel of Desert Ruby’ (U.S. Plant Pat. No. 23,453), ‘Jewel of Desert Garnet’ (U.S. Plant Pat. No. 23,471), ‘Jewel of Desert Moon Stone’ (U.S. Plant Pat. No. 23,491), ‘Jewel of Desert Topaz’ (U.S. Plant Pat. No. 23,492), ‘Jewel of Desert Peridot’ (U.S. Plant Pat. No. 23,566), ‘WOWDRW5’ (U.S. Plant Pat. No. 25,572), ‘WOWDRW7’ (U.S. Plant Pat. No. 25,599), ‘WOWDOY3’ (U.S. Plant Pat. No. 25,600), ‘WOWDAY2’ (U.S. Plant Pat. No. 25,645), ‘WOW2011-1’ (U.S. Plant Pat. No. 25,684), ‘WOWDRY1’ (U.S. Plant Pat. No. 26,157), ‘DSAM13-1’ (U.S. Plant Pat. No. 27,013), ‘DSAA13-1’ (U.S. Plant Pat. No. 27,014) and ‘DSAB13-1’ (U.S. Plant Pat. No. 27,056), and by crossing using the individual mutant, and then selecting said cultivar, (b) a cultivar produced by crossing species, wherein at least either one of the parents of the species is selected from the Variety B, or (c) a progeny plant of one of the cultivars (a) and (b).

In order to solve the aforementioned issues, the present invention is a Delosperma plant obtained by utilizing either one of pollen, ovum, cell, and genetic information relating to DNA and RNA of the abovementioned Delosperma plant.

In order to solve the aforementioned issues, the present invention is a method for producing a Delosperma plant comprising; (a) utilizing either one of pollen, ovum, cell, and genetic information relating to DNA and RNA of the Delosperma plant of claimed above so as to produce the Delosperma plant.

In order to solve the aforementioned issues, the present invention is a breeding method for producing a Delosperma plant comprising; (a) crossing the Delosperma plant of claimed above; and (b) selecting a Delosperma plant having the characteristics from among the progenies of the crossed Delosperma plant.

According to the present invention, the Delosperma plant has more than 150 petals, preferably more than 200 petals, more preferably more than 250 petals, and even more preferably more than 300 petals; therefore, the Delosperma plant has the characteristic of having greater number of petals as compared with conventional cultivars of Delosperma.

### BRIEF DESCRIPTION OF THE DRAWINGS

The present invention will be described hereinafter with reference to the annexed drawing. It is to be noted that the drawing is shown for the purpose of illustrating the technical concepts of the present invention or embodiments thereof, wherein:

FIG. 1 shows Delosperma plants in bloom according to embodiment 1 of the present invention.

FIG. 2 shows Delosperma plants in bloom according to embodiment 1 of the present invention.

FIG. 3 shows Delosperma plants in bloom according to embodiment 1 of the present invention.

FIG. 4 shows Delosperma plants in bloom according to embodiment 1 of the present invention.

FIG. 5 shows Delosperma plants in bloom according to embodiment 1 of the present invention.

FIG. 6 shows Delosperma plants in bloom according to embodiment 1 of the present invention.

FIG. 7 shows a flowchart to show processes of cross breeding.

FIG. 8a and FIG. 8b show a Delosperma plant comprising a flower with more than 150 petals according to embodiment 1 of the present invention.

FIG. 8c is a chart to show the number of petals of the Delosperma plant.

FIG. 9a and FIG. 9b show a Delosperma plant comprising a flower with more than 200 petals according to embodiment 1 of the present invention.

FIG. 9c is a chart to show the number of petals of the Delosperma plant.

FIG. 10a and FIG. 10b show a Delosperma plant comprising a flower with more than 250 petals according to embodiment 1 of the present invention.

FIG. 10c is a chart to show the number of petals of the Delosperma plant.

FIG. 11a and FIG. 11b show a Delosperma plant comprising a flower with more than 300 petals according to embodiment 1 of the present invention.

FIG. 11c is a chart to show the number of petals of the Delosperma plant.

FIG. 12a shows Delosperma sutherlandii.

FIG. 12b and FIG. 12c show the roots of Delosperma sutherlandii.

FIG. 13a shows Delosperma ashtonii.

FIG. 13b and FIG. 13c show the roots of Delosperma ashtonii.

FIG. 14a shows a Delosperma plant in bloom according to embodiment 2 of the present invention.

FIG. 14b shows a photograph showing when outer petals and inner petaloids of the Delosperma plant are separeted.

FIG. 15 shows the Delosperma plant in bloom according to embodiment 2 of the present invention.

FIG. 16a and FIG. 16b show Delosperma plants in bloom according to embodiment 1 of the present invention.

FIG. 17 shows conventional Delosperma cooperi in bloom.

FIG. 18 shows conventional Delosperma “Jewel of Desert Garnet” in bloom.

FIG. 19 shows conventional Delosperma sutherlandii in bloom.

### DETAILED DESCRIPTION OF THE INVENTION

Embodiments of the present invention, as best mode for carrying out the invention, will be described hereinafter with reference to the drawing. It is to be understood that the embodiments described herein are not intended as limiting, or encompassing the entire scope of, the present invention.

### Embodiment 1

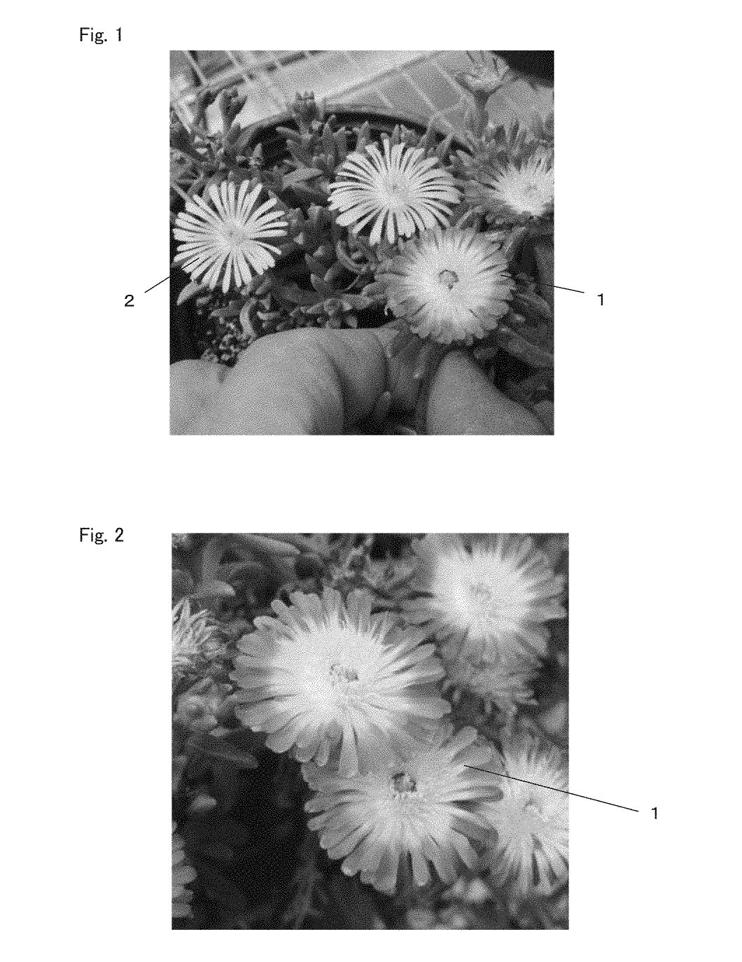

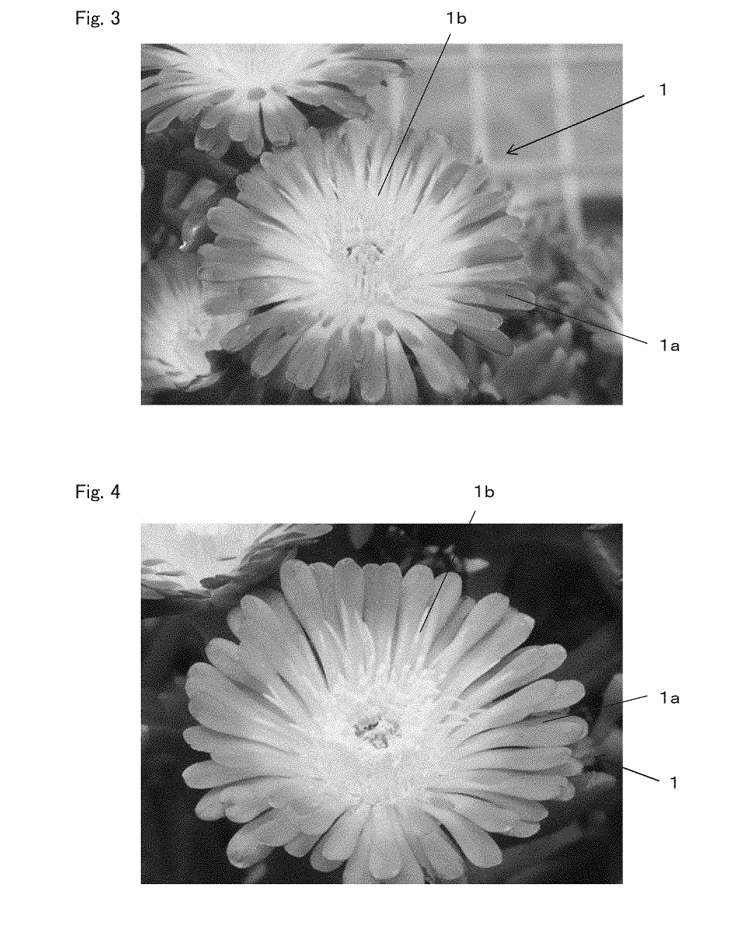

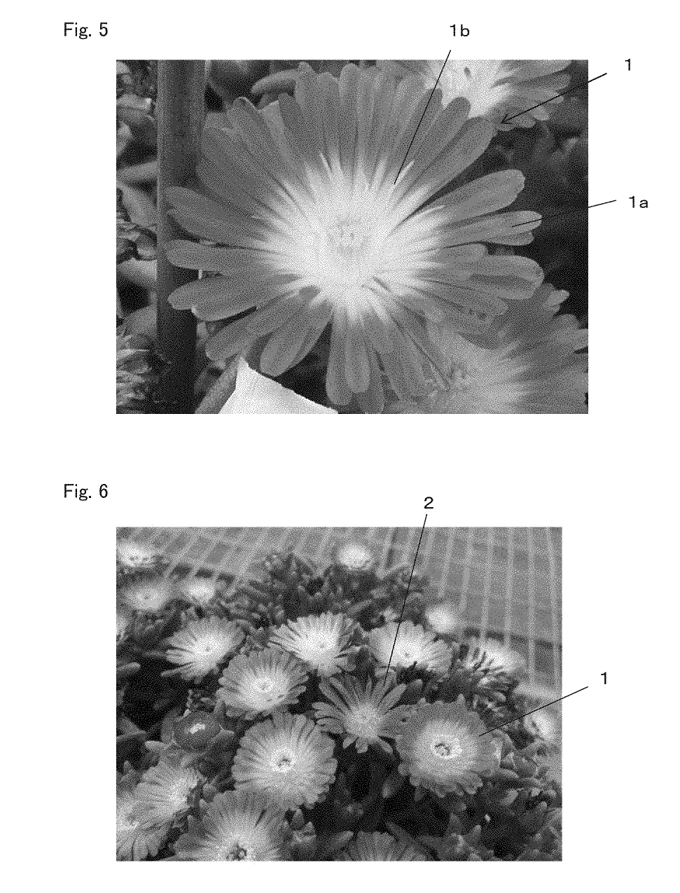

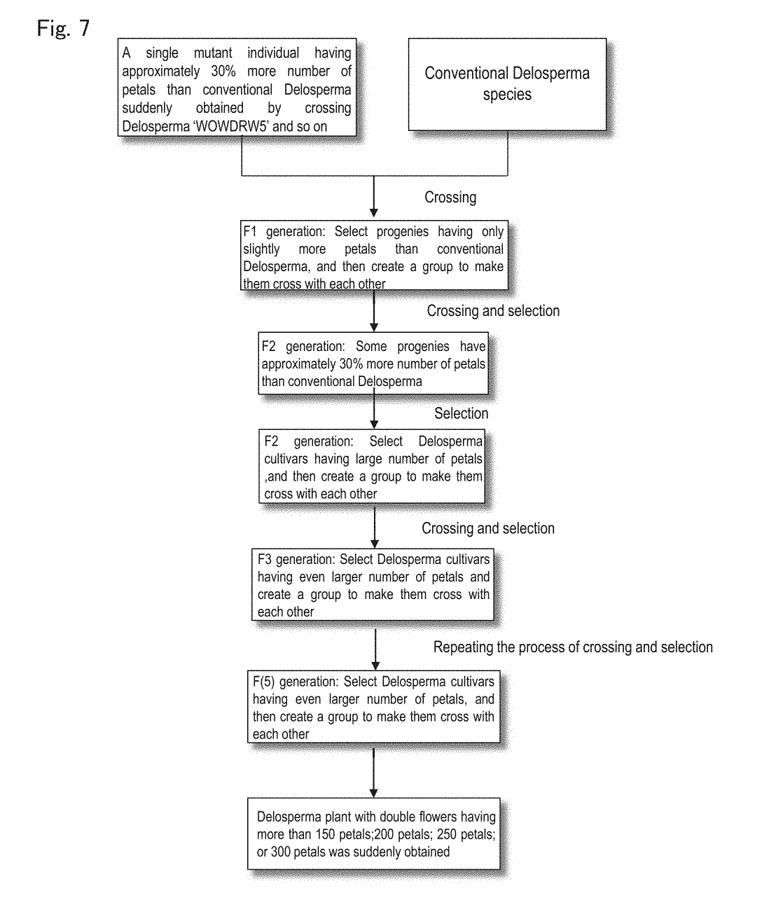

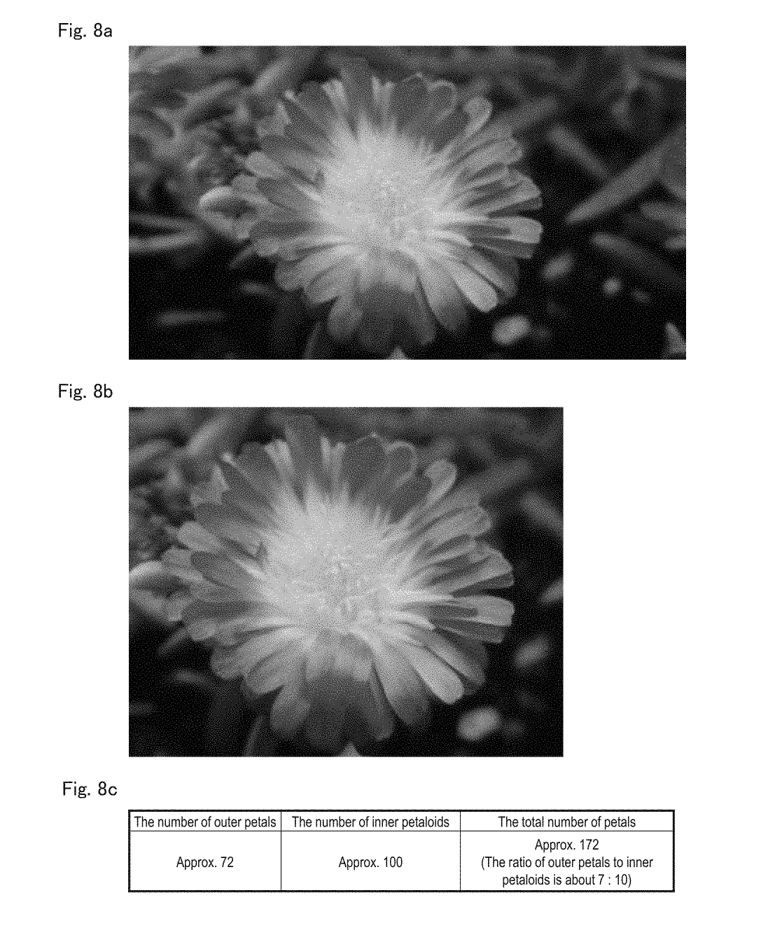

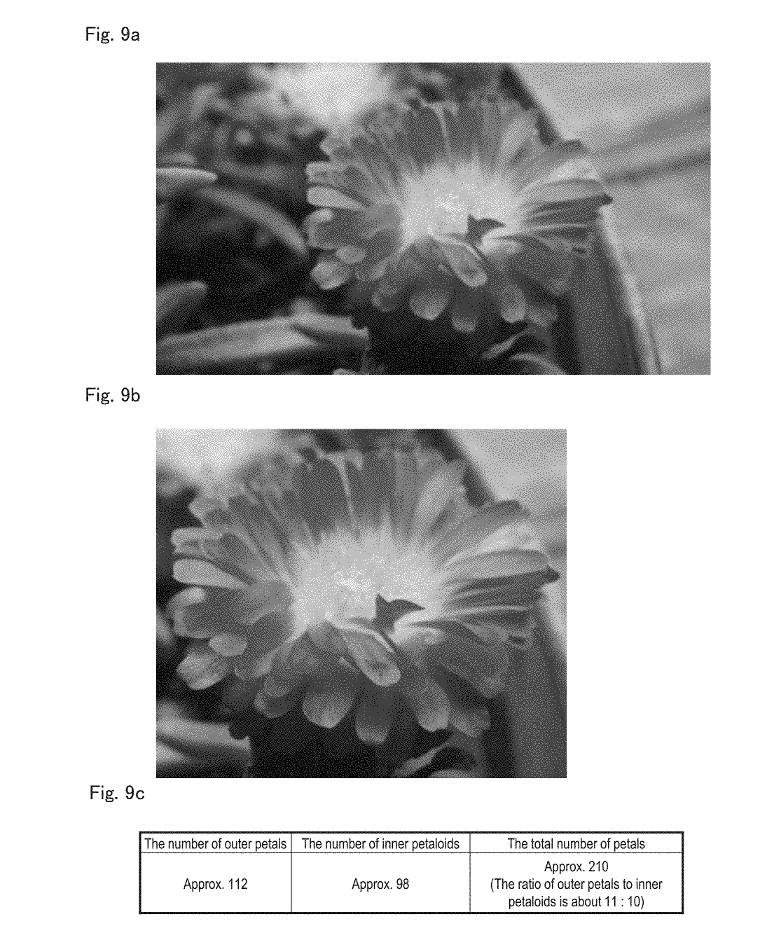

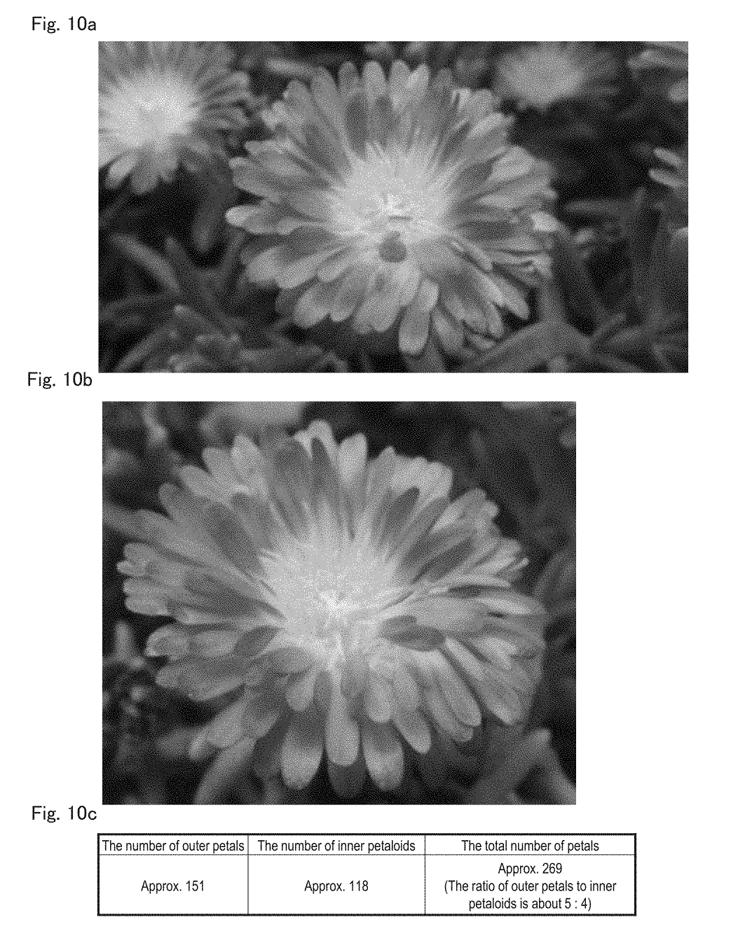

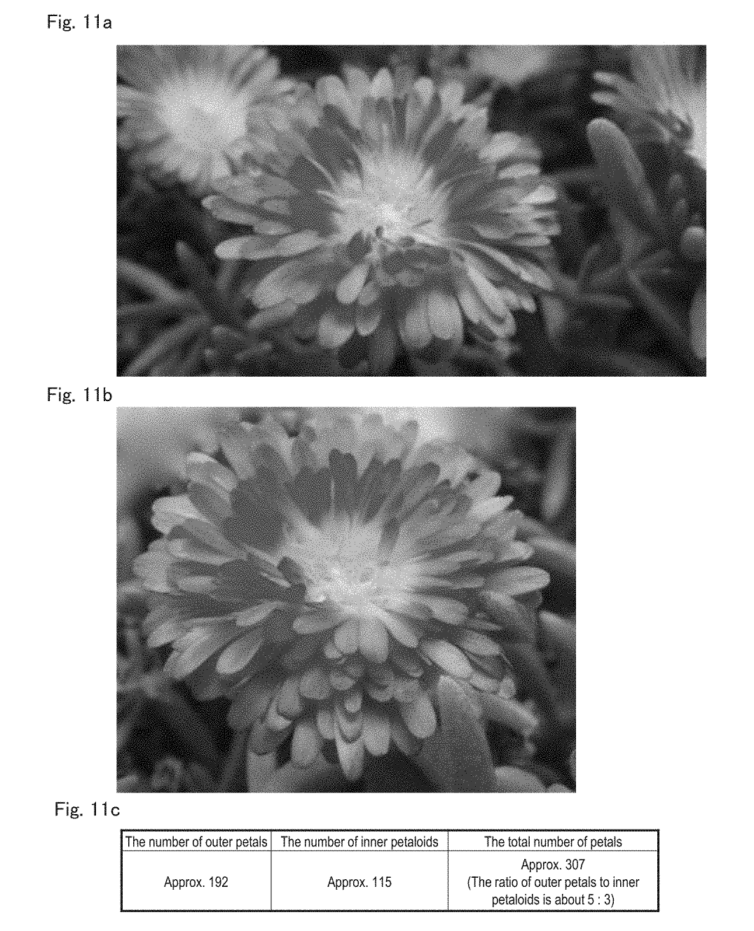

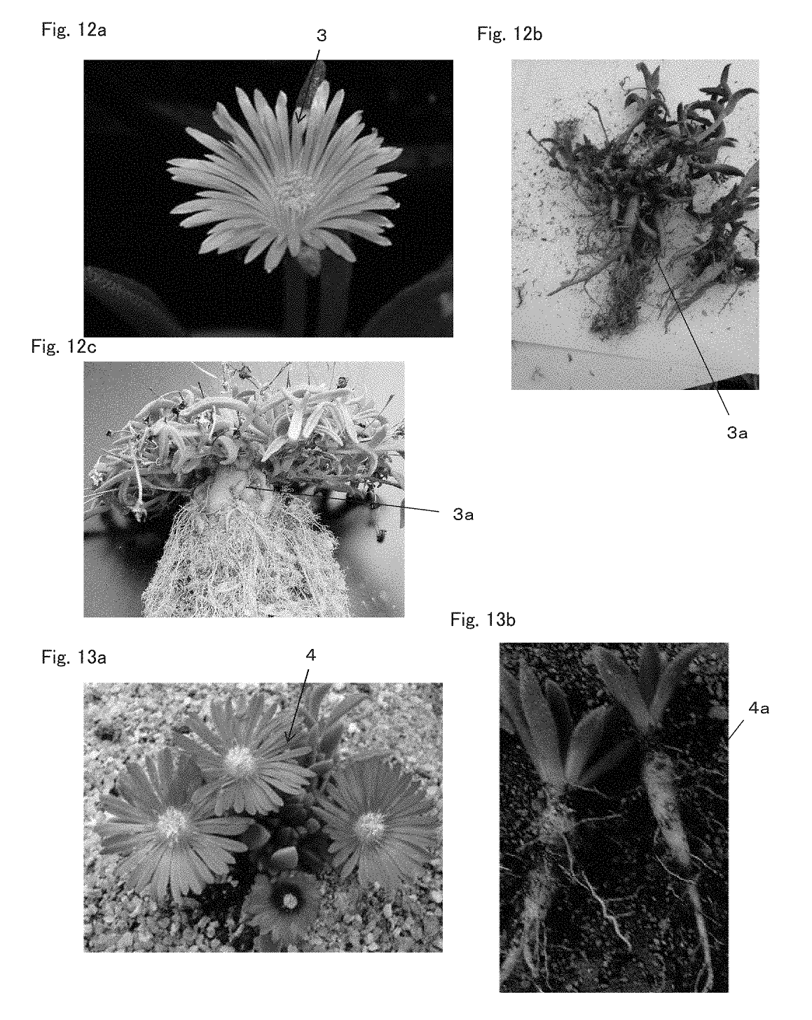

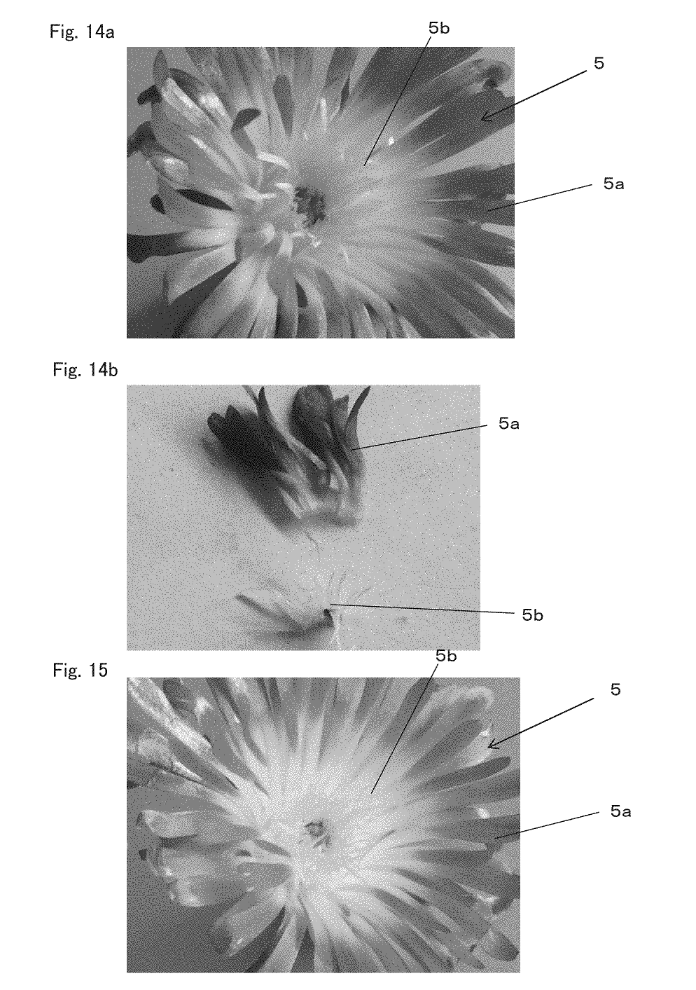

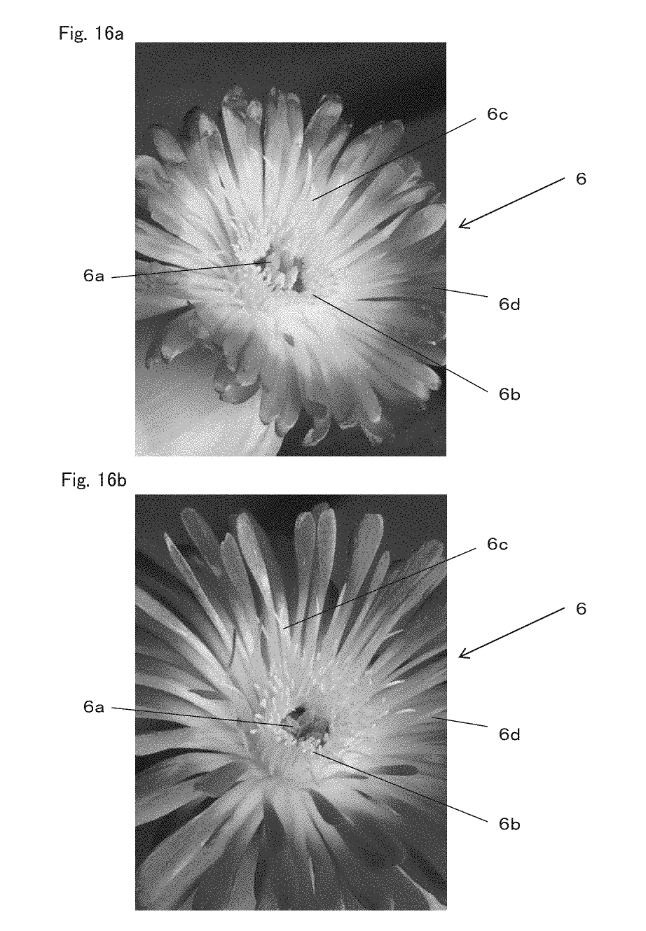

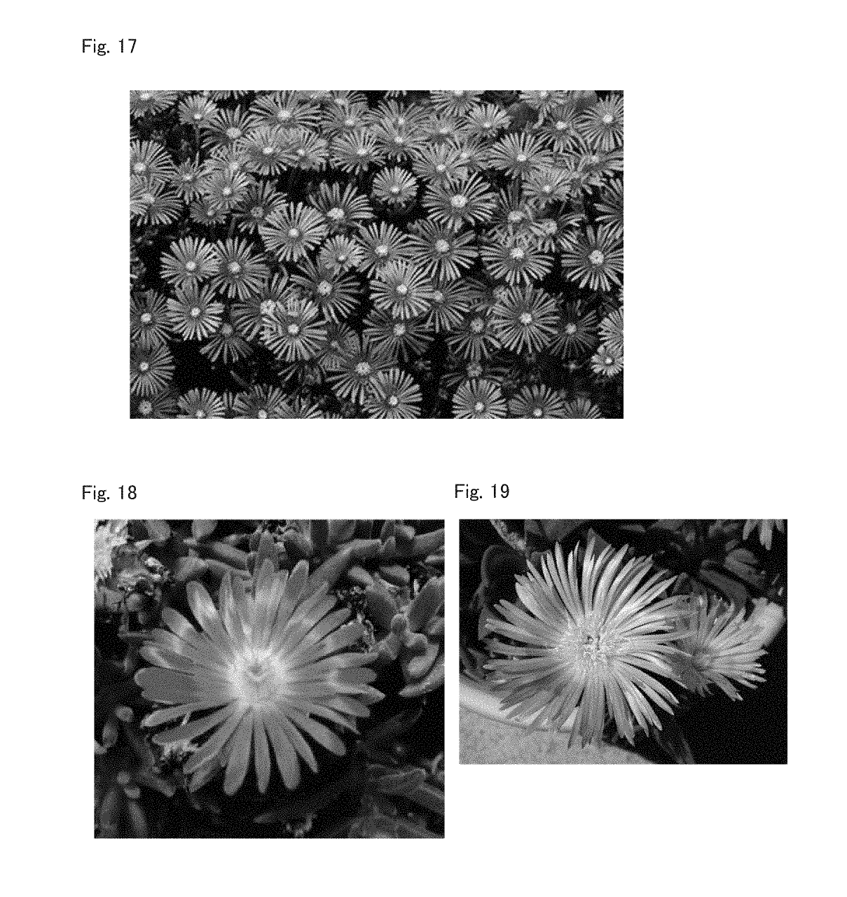

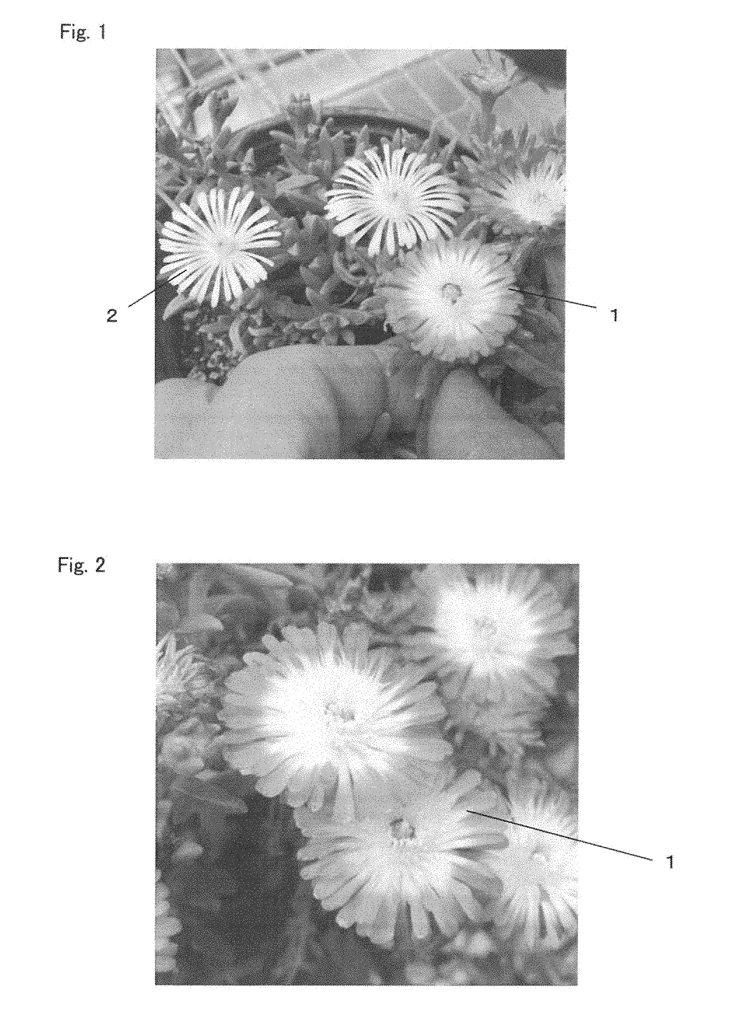

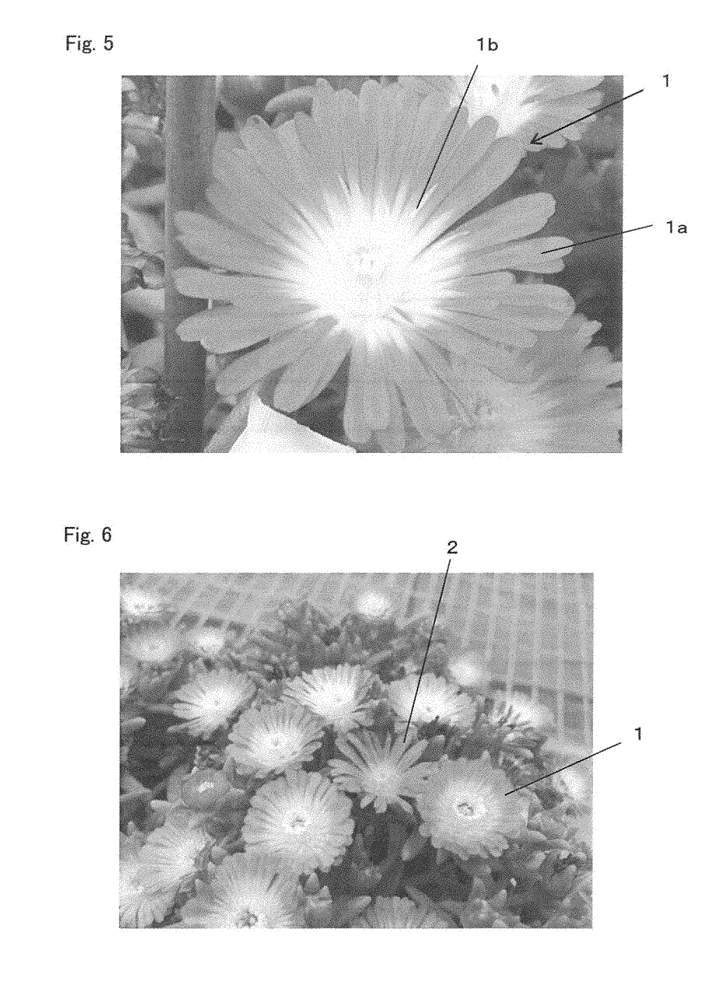

Referring from FIG. 1 to FIG. 13, a Delosperma plant according to Embodiment 1 of the present invention will be described. The inventor of the present application has successfully obtained a plant cultivar shown in FIG. 1 to FIG. 6 (hereinafter referred as “Delosperma 1”), having a characteristic of double flowers, after repeating the process of seeding, crossing and selection. The number of petals of Delosperma 1 shown in FIG. 1 to FIG. 6 is larger (e.g. 150 or more petals) than conventional Delosperma. Herein, Delosperma 2 in FIG. 1 and Delosperma 2 in FIG. 6, which are intentionally inserted for comparison purposes, are conventional cultivars, and it can be recognized that Delosperma 1 has much larger number of petals as compared with the conventional cultivars.

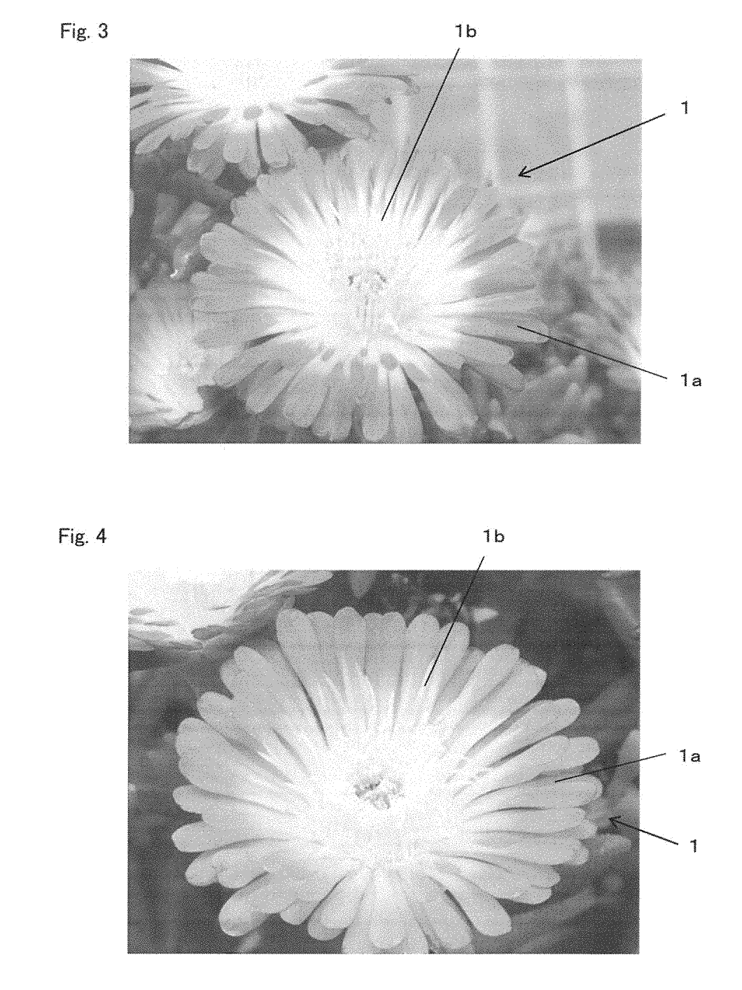

The term “the number of petals” herein consists of outer petals(1a) and inner petaloids around pistil(1b) of the Delosperma plant 1 as indicated in FIG. 3 to FIG. 6. In other words, “the number of petals” includes the number of staminodes (stamens converted into petals) and inner petaloids, which have grown up and become large outer petals, around the pistil

The embodiment 1 of the present invention provides a new Delosperma plant, which is (a) a cultivar called Variety A, which is produced by obtaining an individual mutant from hybridizing at least one of the individuals selected from a group consisting of Delosperma cooperi, Delosperma nubigenum, Delosperma coungestum, Delosperma esterhuysenisa, Delosperma floribundum, Delosperma aberdeenense and Delosperma ‘John Proffitt’, and by crossing using the individual mutant and then selecting said cultivar, (b) a cultivar produced by crossing species, wherein at least either one of the parents of the species is selected from the Variety A, or (c) a progeny of one of the cultivars (a) and (b). Note that these Delosperma species are basically able to cross with each other and able to generate progenies. This crossing includes self-pollination inside an individual plant and cross-pollination between different individual plants. In case of self-pollination, it uses the pistil of an individual plant and pollens collected from the same individual plant. In case of cross pollination, one parent must be the Delosperma plant related to embodiment 1 of the present invention, while the other parent is not necessary to be such the Delosperma plant as long as it is possible for the other parent to cross with the Delosperma plant according to Embodiment 1 and to generate progenies.

Further, a Delosperma plant relating to embodiment 1 herein is (a) a cultivar called Variety B, which is produced by obtaining an individual mutant from hybridizing at least one of the individuals selected from a group consisting of ‘Jewel of Desert Rosequartz’ (U.S. Plant Pat. No. 23,452), ‘Jewel of Desert Ruby’ (U.S. Plant Pat. No. 23,453), ‘Jewel of Desert Garnet’ (U.S. Plant Pat. No. 23,471), ‘Jewel of Desert Moon Stone’ (U.S. Plant Pat. No. 23,491), ‘Jewel of Desert Topaz’ (U.S. Plant Pat. No. 23,492), ‘Jewel of Desert Peridot’ (U.S. Plant Pat. No. 23,566), ‘WOWDRW5’ (U.S. Plant Pat. No. 25,572), ‘WOWDRW7’ (U.S. Plant Pat. No. 25,599), ‘WOWDOY3’ (U.S. Plant Pat. No. 25,600), ‘WOWDAY2’ (U.S. Plant Pat. No. 25,645), ‘WOW2011-1’ (U.S. Plant Pat. No. 25,684), ‘WOWDRY1’ (U.S. Plant Pat. No. 26,157), ‘DSAM13-1’ (U.S. Plant Pat. No. 27,013), ‘DSAA13-1’ (U.S. Plant Pat. No. 27,014) and ‘DSAB13-1’ (U.S. Plant Pat. No. 27,056), and by crossing using the individual mutant, and then selecting said cultivar, (b) a cultivar produced by crossing species, wherein at least either one of the parents of the species is selected from the Variety B, or (c) a progeny plant of one of the cultivars (a) and (b).

A method for producing/breeding the Delosperma plant according to embodiment 1 of the present invention has no particular restriction, except that at least either one of parents should be the Delosperma plant which comprises more than 150 petals, preferably more than 200 petals, more preferably more than 250 petals, and even more preferably more than 300 petals.

Delosperma can be propagated by using seeds, planting cuttings and layering, both in nature and in deliberate cultivation. The Delosperma plant according to embodiment 1 of the present invention can be obtained by utilizing either one of reproduced pollen, ovum, cell, and the genetic information contained in the DNA and RNA of the Delosperma species with abovementioned characteristics. In other words, any reproducible parts of the Delosperma plant can be used for reproducing, and the reproduction is not limited to sexual reproduction but also includes asexual reproduction. Herein, the term “plant(s)” includes plant organs, plant tissues, cells, vegetative propagules and the like, and the plant organs include petals, corolla, flowers, leaves, seeds, fruits, stems, roots, and the like.

The Delosperma plant according to embodiment 1 of the present invention has the characteristics such that; comprising more than 150 petals; preferably more than 200 petals; more preferably more than 250 petals; even more preferably more than 300 petals; and has double flowers having especially a larger number of petals. Note that aforementioned characteristics or a combination of the characteristics of this new Delosperma plant is clearly distinguished from the characteristics of conventional Delosperma species. It is said that the number of petals among conventional Delosperma species is less than 105 at most; therefore, the new Delosperma plant according to embodiment 1 of the present invention with genetical characteristics of having more than 150 petals is a totally new cultivar with different characteristics.

### EXAMPLES

Next, referring to examples below, the Delosperma plant according to Embodiment 1 will be described. It is to be understood that the examples described herein are not intended to limiting the entire scope of the present invention.

### Example 1

The new Delosperma plant relating to the present invention was invented at a farm located in Okayama Prefecture, Japan, under a breeding program instructed and managed by the inventor. The purpose of this breeding program was to breed a new Delosperma plant with a large number of petals, and the number of petals is far larger than conventional Delosperma species. It is noted that the breeding condition of the new Delosperma plant according to the present invention is not particularly limited to the condition described in Example 1, as long as the condition is applicable to the Delosperma plant.

Firstly, the inventor collected 2,000 of hybrid seeds obtained through crossing of Delosperma cultivars selected from a group consisting of ‘Jewel of Desert Rosequartz,’ ‘Jewel of Desert Ruby,’ ‘Jewel of Desert Garnet,’ ‘Jewel of Desert Moon Stone,’ ‘Jewel of Desert Topaz,’ ‘Jewel of Desert Peridot,’ ‘WOW(Wheels of Wonder)DRW5,’ ‘WOWDRW7,’ ‘WOWDOY3,’ ‘WOWDAY2,’ ‘WOW2011-1,’ ‘WOWDRY1,’ ‘DSAM13-1,’ ‘DSAA13-1’ and ‘DSAB13-1’ owned by the inventor, and bred them in a greenhouse near the inventor's house located at Katsuta-gun in Okayama Prefecture, Japan, with ordinary breeding methods. After having crossed the abovementioned seeds with various seeds owned by the inventor for three generations repeatedly, a single mutant individual of a Delosperma plant with a unique characteristic, which has approximately 30% more number of petals than conventional Delosperma, was suddenly obtained.

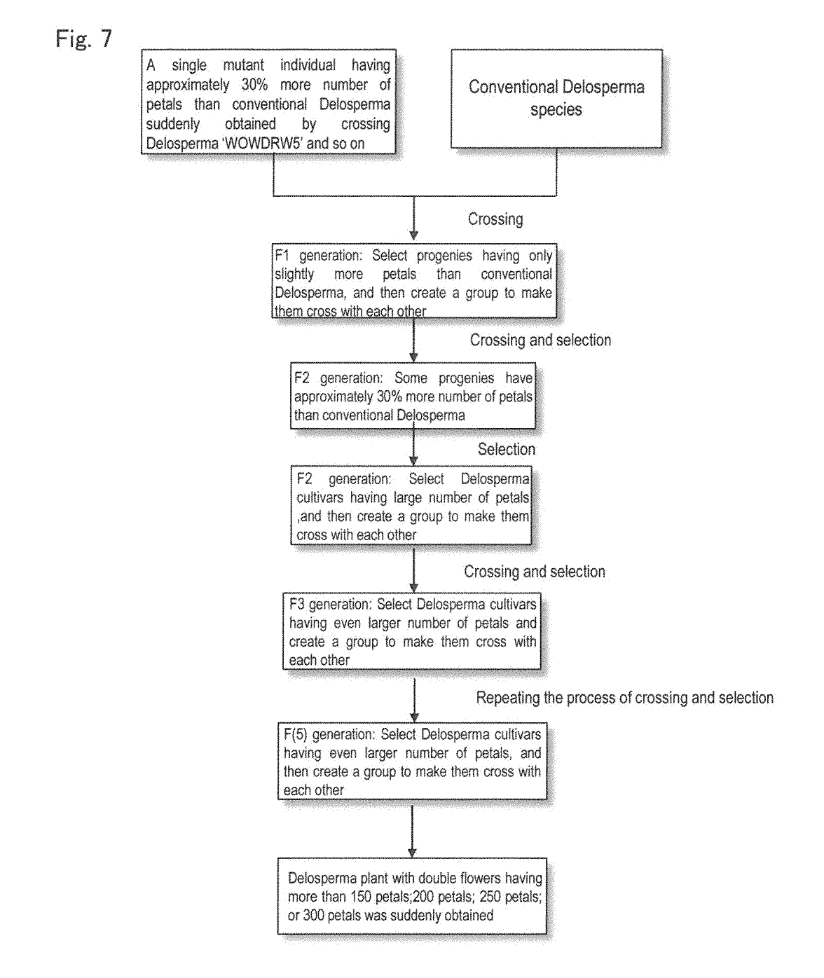

Next, after having repeated crossing and selection processes using the cultivars from a group of progenies obtained from the single mutant individual, a Delosperma plant having a large number of petals was successfully obtained. To be more specifically, as described in FIG. 7, the procedure is as follows: (1) crossing the mutant individual of Delosperma, which has approximately 30% more petals than its parents. The mutant Delosperma plant was suddenly obtained by crossing Delosperma species selected from a group of Delosperma ‘WOWDRW5’ and so on; (2) next, collecting seeds from the Delosperma plant, and selecting Delosperma cultivars having even larger number of petals obtained by breeding the seeds. And crossing said Delosperma cultivars, and then selecting Delosperma cultivars having larger number of petals (repeating crossing and selection) for multiple generations in order to gradually enhance the characteristic of having a large number of petals. However, it is noted that when Delosperma cultivars are grown from seeds, the number of petals of Delosperma is not stable. For example, the ratio of cultivars with a large number of petals was sometimes below 10% and sometimes over 30% in the next generation. When reaching a stage that some of Delosperma cultivars have a large number of petals, a Delosperma plant with double flowers having more than 150 petals, preferably having more than 200 petals, more preferably more than 250 petals, and even more preferably more than 300 petals was suddenly emerged. And (3) genetically fixing a desired characteristic (having more than 150 petals) by repeating crossing and selection of the Delosperma plant having said characteristic even further for multiple generations. In other words, the Delosperma plant with the desired characteristic can surely be obtained, even in small amount, by utilizing the crossed Delosperma plant (as at least either one of the parents for crossing) within multiple generations (which means that the characteristic is repeatable).

As described above, the characteristic of the Delosperma plant according to embodiment 1 of the present invention is “repeatable(inheritable)”, which means that the same result can be repeatable(inheritable) if the same breeding method is repeated, and the Delosperma plant is clearly different from a conventional plant in its characteristic (a number of petals). In addition, the invertor has already obtained more than 100 double-flower type Delosperma species having especially a large number of petals by utilizing the Delosperma plant, which was initially obtained and was a double-flower type specie having a large number of petals. This also indicates that the characteristic according to the present invention is inheritable.

### Example 2

Secondly, a crossing experiment was implemented. Specifically, the Delosperma plant with the specific characteristic herein had been bred and the pollens from stamens of Delosperma with the usual number of petals were deposited into a pistil of said Delosperma plant. The Delosperma plant was bred until it generated seeds, and then the seeds collected from the plant were sowed and bred. As a result, double-flower type species having a especially large number of petals which have more than 150 petals, preferably more than 200 petals, more preferably more than 250 petals and even more preferably more than 300 petals were found in the first generation(a child generation), the next generation(a grandchild generation), and the next generation obtained through the said breeding procedures.

Herein, the ratio of a double-flower type specie having a large number of petals was sometimes below 10% and sometimes over 30%.

Accordingly, by means of depositing pollens from fertile commercial cultivar into the pistil of the double-flower type Delosperma plant having a large number of petals according to embodiment 1 of the present invention, the double-flower type Delosperma having a large number of petals will be produced in following generations, and hence this shows that this characteristic is repeatable. As described above, in Example 2, the Delosperma plant with the desired characteristics can surely be obtained by utilizing the Delosperma plant (as at least either one of the parents for crossing) within multiple generations.

### Example 3

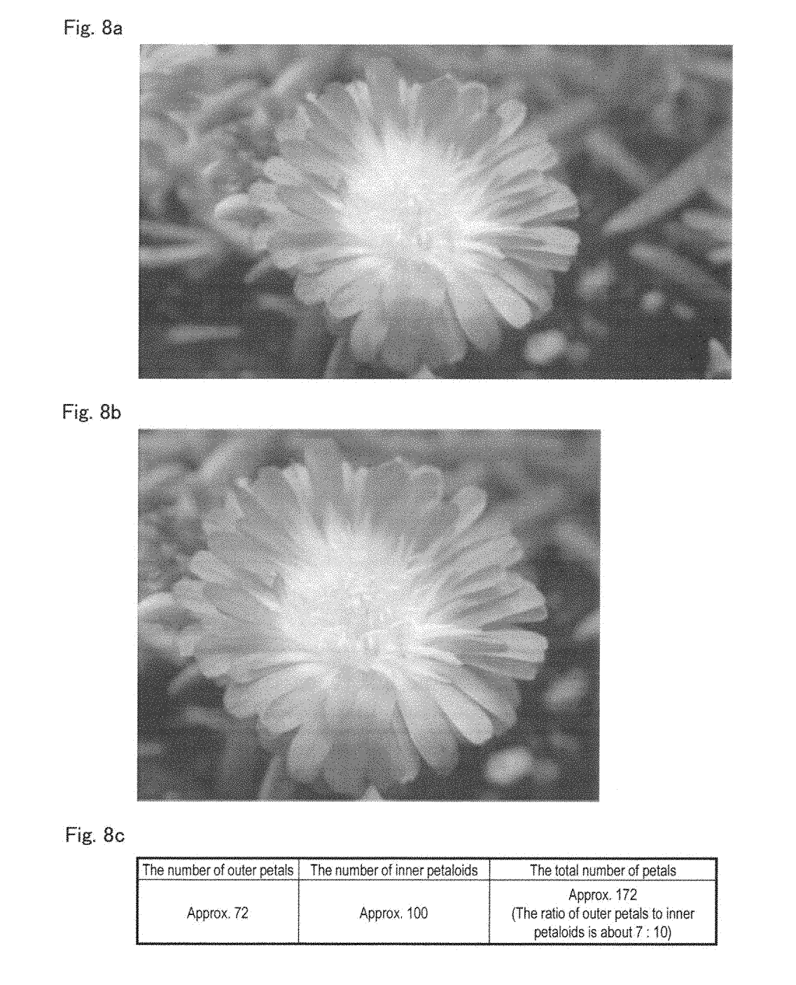

The details of the double-flower type Delosperma plant having a large number of petals will be explained as follows. FIG. 8(a) and FIG. 8(b) show the Delosperma having more than 150 petals according to Example 3, which has approximately 172 numbers of petals in total consisted of around 72 outer petals and around 100 inner petaloids as described in FIG. 8 (c). The ratio of outer petals to inner petaloids is about 7:10. However, it is noted that the ratio of outer petals to inner petaloids is not particularly limited, and can be 6:4, 6:5, or 6:3.

### Example 4

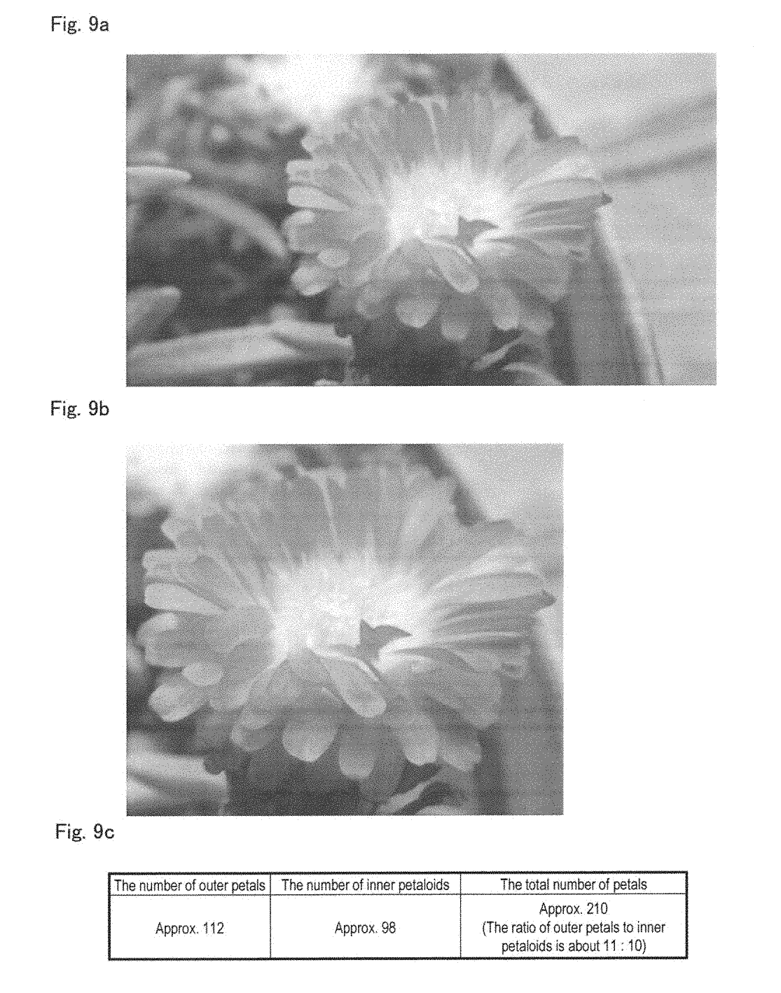

FIG. 9 shows the Delosperma plant having more than 200 petals according to Example 4, which has approximately 210 numbers of petals in total consisting of around 112 outer petals and around 98 inner petaloids. The ratio of outer petals to inner petaloids is about 11:10.

### Example 5

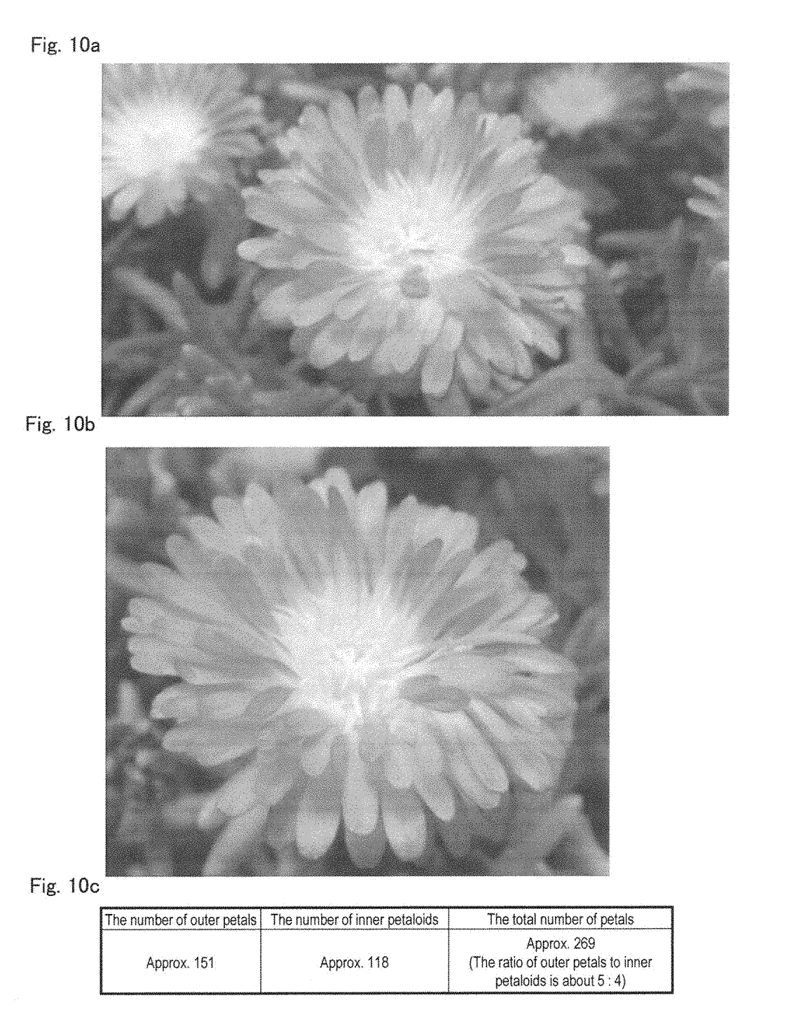

FIG. 10 shows the Delosperma plant having more than 250 petals related to Example 5, which has approximately 269 numbers of petals in total consisting of around 151 outer petals and around 118 inner petaloids. The ratio of outer petals to inner petaloids is about 5:4.

### Example 6

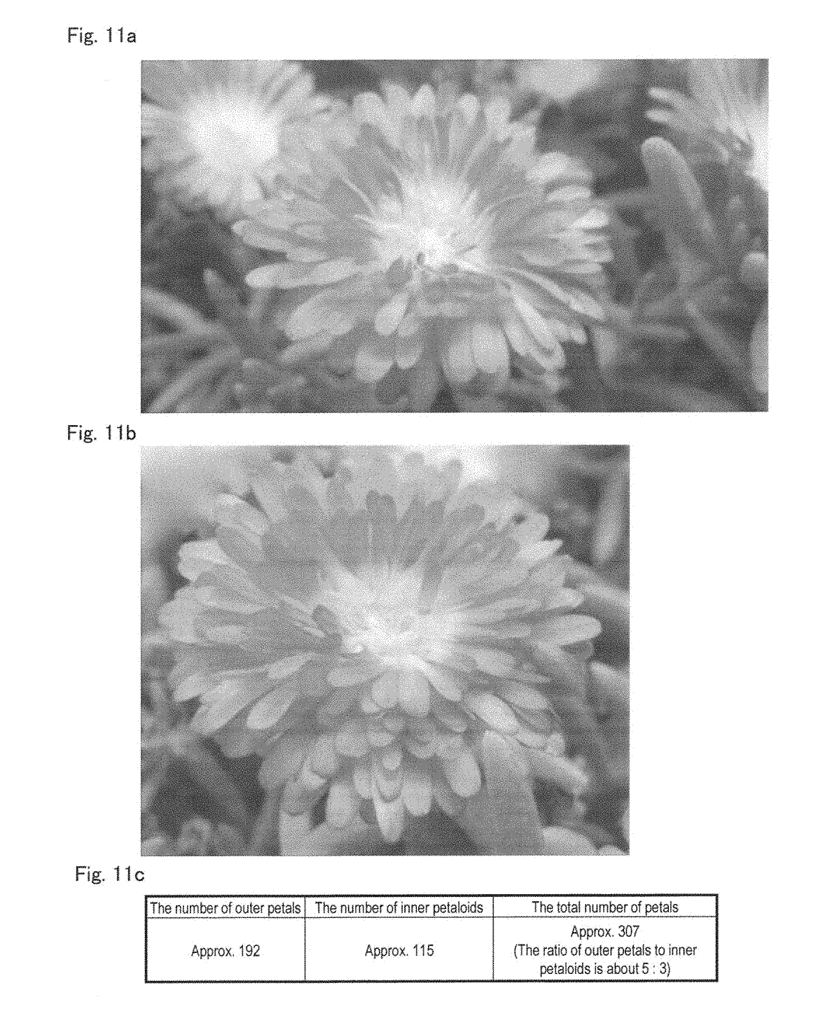

FIG. 11 shows the Delosperma plant having more than 300 petals related to Example 6, which has approximately 307 numbers of petals in total consisting of around 192 outer petals and around 115 inner petaloids. The ratio of outer petals to inner petaloids is about 5:3.

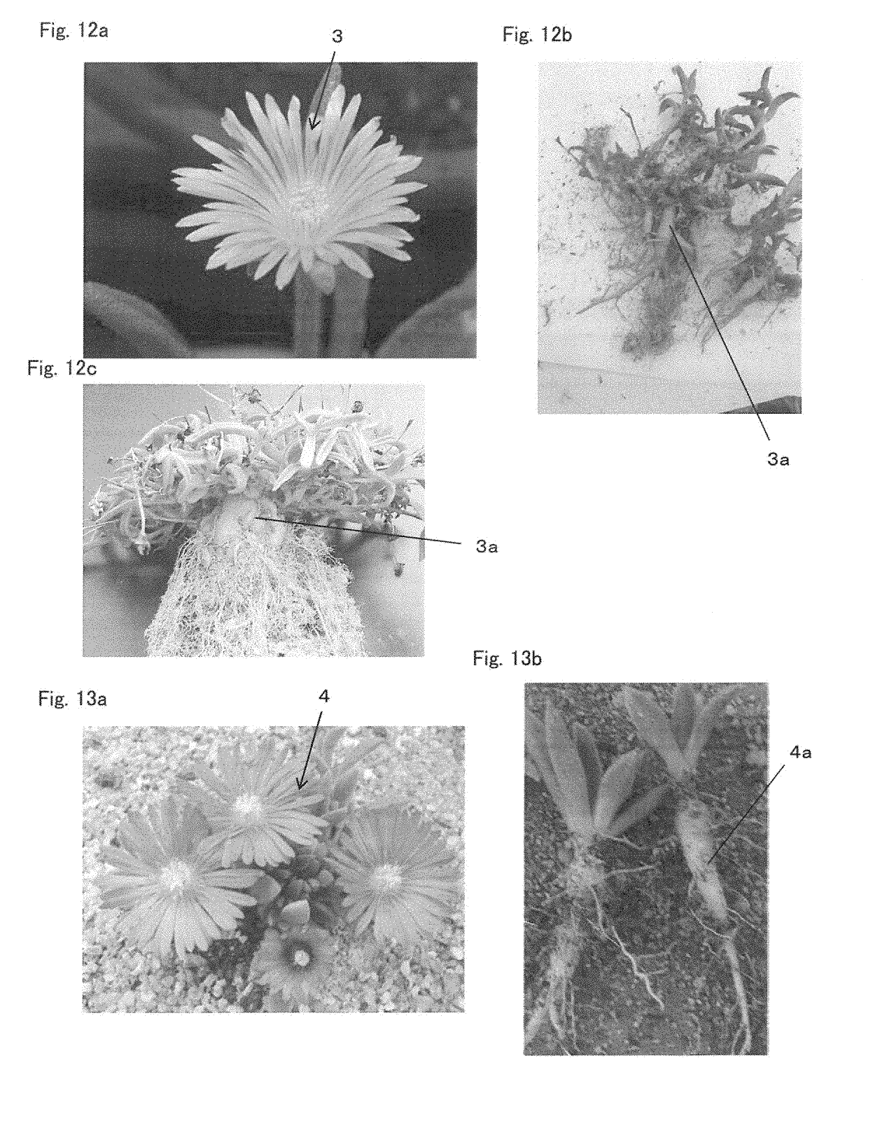

Next, the details of roots of the Delosperma plant according to embodiment 1 of the present invention will be explained, referring to FIG. 12 and FIG. 13. Because, the root parts of the Delosperma plant are totally different from those of Delosperma sutherlandii and Delosperma ashtonii, which have relatively large number of petals.

The Delosperma plant according to embodiment 1 of the present invention is also characterized by its roots which becomes capillary roots and does not form tuberous roots. On the other hand, as shown in FIG. 12 (b), FIG. 12 (c), and FIG. 13 (b), Delosperma sutherlandii 3 and Delosperma ashtonii 4 have tuberous roots 3a,4a just like carrot so as to retain nutrition and water in it. Such cultivars sometimes have a relatively large number of petals (e.g. less than 105 petals) and listed in the Delosperma cultivar at present. However, the structure of root parts of such cultivars is obviously different from the Delosperma plant according to embodiment 1 of the present invention.

Furthermore, Delosperma cultivars which forms tuberous roots such as Delosperma sutherlandii 3 are attractive because of its relatively large number of petals and voluminous size. However, because of its heat intolerance and significant extreme sensitivity to humidity, such Delosperma cultivars can not be used for the purpose of producing new developed Delosperma species. Moreover, hybrid species were not able to be collected during the crossing experiment between the Delosperma plant relating to embodiment 1 of the present invention and Delosperma sutherlandii 3. Although the Delosperma plant according to embodiment 1 of the present invention botanically belongs to the same variety with Delosperma sutherlandii 3 and Delosperma ashtonii 4, it cannot be used for crossing with them, and the characteristic of root parts is obviously different as described above. It means that, genetic information of Delosperma cultivars which form tuberous roots such as Delosperma sutherlandii 3 and Delosperma ashtonii 4 are not used for producing the Delosperma plant herein.

As described above, the Delosperma plant according to embodiment 1 of the present invention has a large number of petals, such as more than 150 petals, as compared with conventional cultivars (approximately twice as much), and forms the structure of double flowers having especially large number of petals. As a result, the present invention can provides the new Delosperma plant that is more voluminous and more commercially valuable than conventional Delosperma. It is to be understood that the abovementioned Delosperma plant described herein is not intended to limiting the scope of morphological and ecological characteristics of Delosperma, except for the number of petals and the characteristic of root parts.

### Embodiment 2

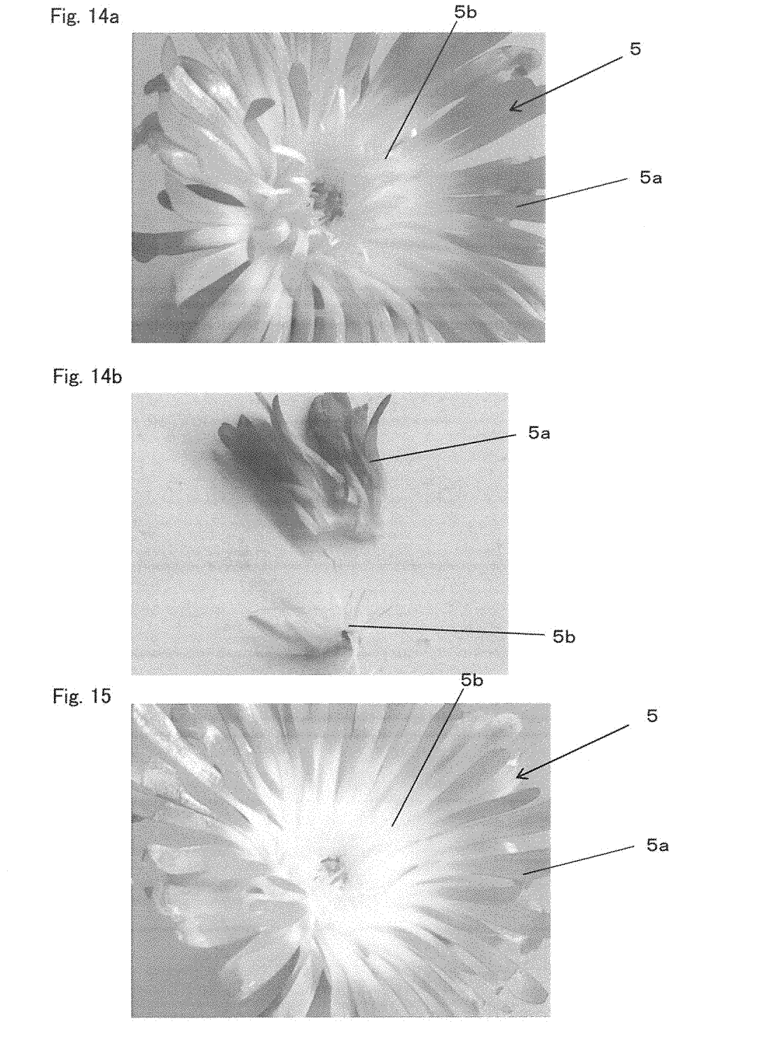

Referring from FIG. 14 to FIG. 16, a Delosperma plant according to Embodiment 2 of the present invention will be described.

The following characteristics have been represented and observed at the Delosperma plant according to embodiment 2 of the present invention. The Delosperma plant described herein has the characteristics of not having stamens with anther in addition to having a large number of petals. On the other hand, conventional Delosperma species usually have several tens of stamens. Therefore, it can be said that the Delosperma plant according to embodiment 2 of the present invention with characteristics of having a large number of petals and not having stamens with anther is a new cultivar with completely new characteristics. These characteristics and the combination of these characteristics are clearly distinguished as a new Delosperma variety from the conventional Delosperma varieties.

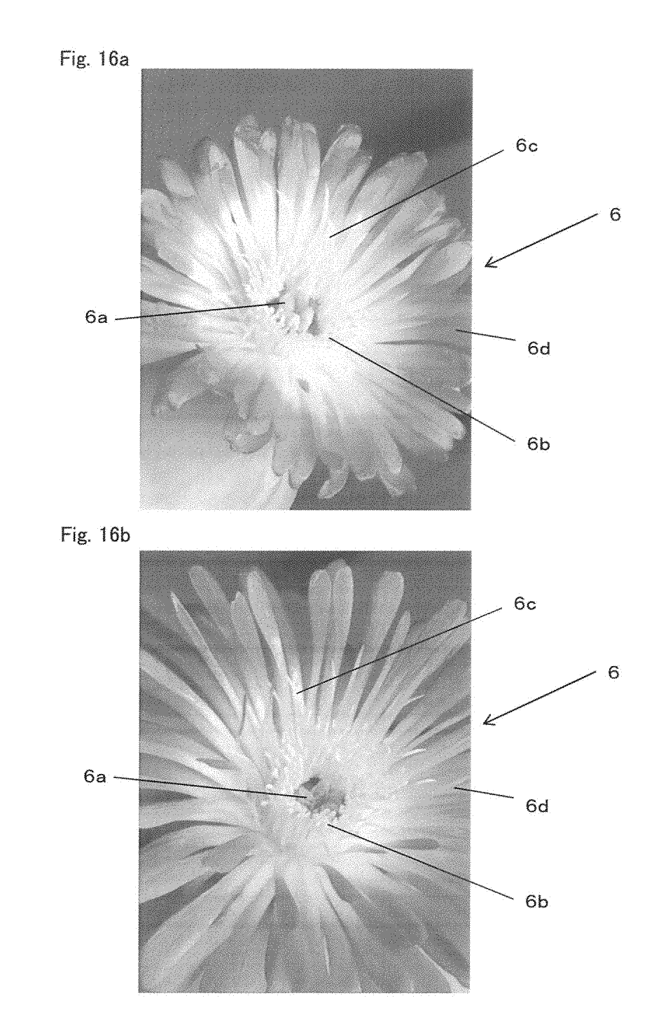

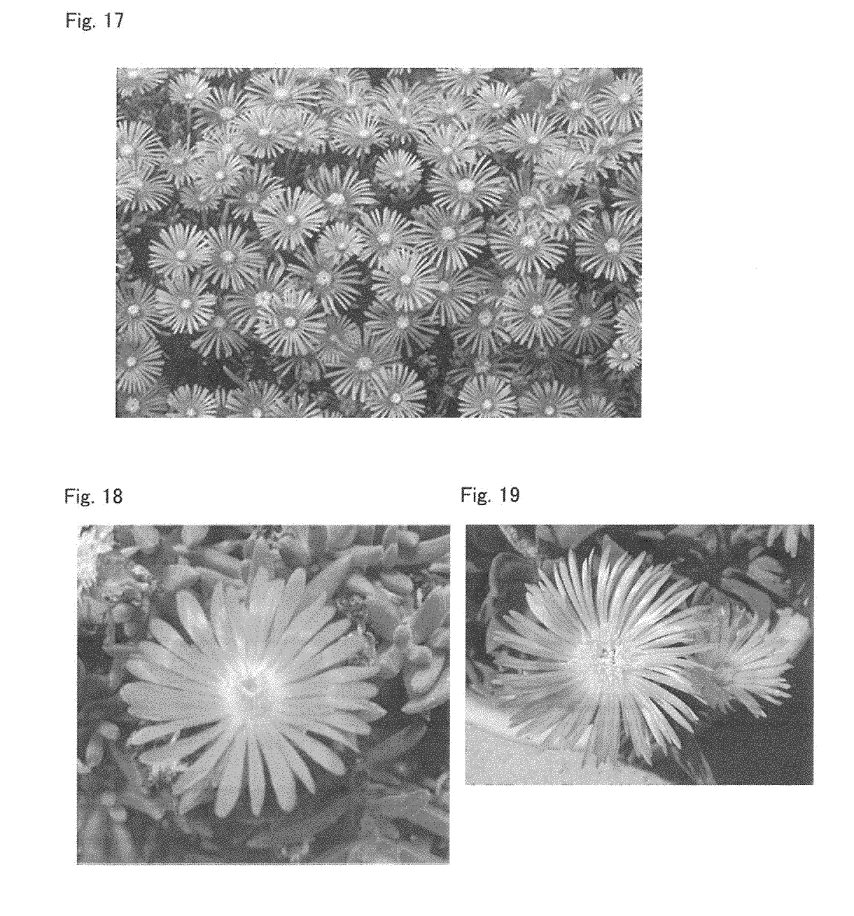

The double-flower type Delosperma species having especially a large number of petals and not having stamens with anther (hereinafter referred to as “Delosperma 5”) are shown in FIG. 14 and FIG. 15. It can be recognized that all stamens of Delosperma 5 are converted and grown into petals 5a and petaloids 5b. On the other hand, it can be observed that, the Delosperma plant according to embodiment 1 of the present invention in FIG. 16 (hereinafter referred to as “Delosperma 6”), which is represented for comparison purposes, has a pistil 6a, stamens 6b, petals 6c and petaloids 6d, in order from the center toward the outer end of the flower of Delosperma 6.

Because the Delosperma plant described herein has far larger number of petals than conventional Delosperma species, it is more commercially valuable. In addition to this, since the Delosperma plant is a male sterile plant, which can avoids seeds from being produced, and therefore the Delosperma plant has larger number of blooms. As a result of this, it is clear that the Delosperma plant according to embodiment 2 has more commercial values.

It is to be understood that the abovementioned Delosperma plant described herein is not intended to limiting the scope of morphological and ecological characteristics of Delosperma, except for the number of petals, having no stamen, and the characteristic of root parts.

### Example

An example of the Delosperma plant according to the second embodiment 2 of the present invention will be explained as follows. However, it is noted that the form of present invention is not limited to the described practical example.

Similar to practical examples described in embodiment 1 of the present invention, the selection procedure is as follows: (1) crossing above mentioned mutant individual of Delosperma, which has approximately 30% more petals than its parents. (2) next, collecting seeds, and selecting Delosperma cultivars having even larger number of petals obtained by breeding the seeds. And crossing said Delosperma cultivars, and then selecting Delosperma cultivars having larger number of petals (repeating crossing and selection) for multiple generations in order to gradually enhance the characteristic of having a large number of petals. In this example, the Delosperma cultivars with characteristics of not having (or having a fewer) stamens with anther in addition to having larger number of petals were selected.

It has been observed that, because this Delosperma plant is a male sterile plant that has no stamen and no anther, the seeds are not likely to be produced unless otherwise a non-self pollen has been deposited from another Delosperma plant.

It is to be noted that the Delosperma plant can be proliferated (asexually reproduced) with herbaceous cutting and other techniques. Herbaceous cutting can be conducted with known methods for a person having ordinary skill in the art. For example, after a tissue cut from Delosperma have been cultivated under the optimal environments in order to produce a plantlet having roots and shoots, and then the plantlet is raised under the environments. The tissue can also be cultivated through mericloning which extracts approximately 1 millimeter of growing point from sprout and cultivates it in sterile culture.

As described above, the Delosperma plant according to embodiment 2 of the present invention has far larger number of petals than conventional cultivar and does not have stamens. Therefore, its flowers get voluminous and the number of blooms per stem increases, because the Delosperma plant is a male sterile plant, and its appearance becomes more contact. Hence, these characteristics can enhance the commercial value of the Delosperma plant.

### Embodiment 3

A Delosperma plant according to Embodiment 3 of the present invention will be described. The Delosperma plant according to embodiment 3 of the present invention has an infertile pistil and unable to generate seeds even when the pistil receives fertile pollens. The self-incompatibility, the reaction that a pistil rejects self pollen to avoid inbreeding, is generally known. However, the Delosperma plant described herein is unable to generate seeds through receiving self pollens (self-pollination) and non-self pollens (external pollination). In other words, the Delosperma plant has the characteristic of a female sterile flower, which is unable to generate seeds even when the pistil receives self or non-self pollens. As a result of this, the Delosperma plant comprises a female sterile flower has more numbers of bloom per stem, and this characteristic makes the Delosperma plant more commercially valuable. Herein, the Delosperma plant mentioned in embodiment 2 of the present invention has no pollen in its stamens, but the pistil can have fertility, and can generate seeds if pollens are deposited from an external plant by chance. On the other hand, the Delosperma plant according to embodiment 3 of the present invention has a female sterile flower having an infertile pistil. Therefore, seeds will never be generated even when it receives any type of pollens.

Such a female sterility is also resulted from mutation and such a characteristic has been enhanced and fixed by repeating crossing and selection for multiple generations. Such a characteristic will emerge at the grandchild generation (e.g. for around 10%) at the experimental station of the inventor.

The present invention is not limited to the configuration of the above embodiments and various modifications can be made within a scope not changing the gist of the present invention. For example, it is possible to breed double-flower type Delosperma having especially larger number of petals and colorful flowers by crossing the Delosperma plants according to the above embodiments with Delosperma cultivars having a flower with different colors.

If bred under harsh environmental conditions for Delosperma, such as excessively humid environment, high temperature environment, and extremely cold environment, the number of petals may decrease. However, the number of petals is restored after being kept under optimal breeding conditions for a while. It means that, depending on the conditions, Delosperma may have flowers with small numbers of petals, however, after being kept under appropriate conditions for a while, it will have flowers with true number of petals. This phenomenon is not limited to the Delosperma specie but can also be found in every plant in general.

Furthermore, it can be expected that genes in the DNA and RNA relating to the characteristics of a large number of petals, having no stamens or a female sterility will be extracted from abovementioned Delosperma plants and embedded the genes into other cells. This will lead to reproducing other plants acquiring such DNA and RNA, and to creating plants having these characteristics other than Delosperma.

1. A Delosperma plant comprising a flower with more than 150 petals, wherein the number of the petals consists of the number of outer petals and the number of inner petaloids around a pistil of the flower.

2. The Delosperma plant of claim 1, wherein the flower includes more than 200 petals.

3. The Delosperma plant of claim 2, wherein the flower includes more than 250 petals.

4. The Delosperma plant of claim 3, wherein the flower includes more than 300 petals.

5. The Delosperma plant of claim 1, wherein the Delosperma plant comprises no stamen.

6. The Delosperma plant of claim 1, wherein the Delosperma plant comprises a female sterile flower, which is unable to generate seeds even when the pistil receives self or non-self pollens.

7. The Delosperma plant of of claim 1, wherein the Delosperma plant forms no tuberous root.

8. The Delosperma plant of claim 1, wherein the Delosperma plant is
(a) a cultivar called Variety A, which is produced by obtaining an individual mutant from hybridizing at least one of the individuals selected from a group consisting of Delosperma cooperi, Delosperma nubigenum, Delosperma coungestum, Delosperma esterhuysenisa, Delosperma floribundum, Delosperma aberdeenense and Delosperma ‘John Proffitt’, and by crossing using the individual mutant and then selecting said cultivar,
(b) a cultivar produced by crossing species, wherein at least either one of the parents of the species is selected from the Variety A, or
(c) a progeny of one of the cultivars (a) and (b).

9. The Delosperma plant of claim 1, wherein the Delosperma plant is
(a) a cultivar called Variety B, which is produced by obtaining an individual mutant from hybridizing at least one of the individuals selected from a group consisting of ‘Jewel of Desert Rosequartz’ (U.S. Plant Pat. No. 23,452), ‘Jewel of Desert Ruby’ (U.S. Plant Pat. No. 23,453), ‘Jewel of Desert Garnet’ (U.S. Plant Pat. No. 23,471), ‘Jewel of Desert Moon Stone’ (U.S. Plant Pat. No. 23,491), ‘Jewel of Desert Topaz’ (U.S. Plant Pat. No. 23,492), ‘Jewel of Desert Peridot’ (U.S. Plant Pat. No. 23,566), ‘WOWDRW5’ (U.S. Plant Pat. No. 25,572), ‘WOWDRW7’ (U.S. Plant Pat. No. 25,599), ‘WOWDOY3’ (U.S. Plant Pat. No. 25,600), ‘WOWDAY2’ (U.S. Plant Pat. No. 25,645), ‘WOW2011-1’ (U.S. Plant Pat. No. 25,684), ‘WOWDRY1’ (U.S. Plant Pat. No. 26,157), ‘DSAM13-1’ (U.S. Plant Pat. No. 27,013), ‘DSAA13-1’ (U.S. Plant Pat. No. 27,014) and ‘DSAB13-1’ (U.S. Plant Pat. No. 27,056), and by crossing using the individual mutant and then selecting said cultivar,
(b) a cultivar produced by crossing species, wherein at least either one of the parents of the species is selected from the Variety B, or
(c) a progeny plant of one of the cultivars (a) and (b).

10. A Delosperma plant obtained by utilizing either one of pollen, ovum, cell, and genetic information relating to DNA and RNA of the Delosperma of claim 1.

11. A method for producing a Delosperma plant comprising;
(a) utilizing either one of pollen, ovum, cell, and genetic information relating to DNA and RNA of the Delosperma plant of claim 1 so as to produce the Delosperma plant.

12. A breeding method for producing a Delosperma plant comprising;
(a) crossing the Delosperma plant of claim 1; and
(b) selecting a Delosperma plant having the characteristics of the Delosperma plant of claim 1 from among the progenies of the crossed Delosperma plant.
